(19) United States
(12) Patent Application Publication
(10) Pub. No.: US 2016/0058612 A1
(43) Pub. Date: Mar. 3, 2016

(54) Non-Aggravating Thermal Treatment Packs

(71) Applicant: Impact Healthcare Marketing LLC, Monrovia, CA (US)

(72) Inventors: Kyle Ezold, San Marino, CA (US); Andrew Ezold, San Marino, CA (US)

(73) Assignee: Impact Healthcare Marketing LLC, Monrovia, CA (US)

(21) Appl. No.: 14/839,426

(22) Filed: Aug. 28, 2015

Related U.S. Application Data
(60) Provisional application No. 62/043,264, filed on Aug. 28, 2014.

(51) Int. Cl. A61F 7/02; A61F 7/10

(52) U.S. Cl. CPC A61F 7/02; A61F 7/10; A61F 2007/108; A61F 2007/0219; A61F 2007/0222; A61F 2007/0228

(57) ABSTRACT

Systems and methods in accordance with embodiments of the invention implement non-aggravating thermal treatment packs. In one embodiment, a non-aggravating thermal treatment pack includes: a vinyl-based encasing; and a gelatinous material disposed within the vinyl-based encasing, itself including: a solute that is a Cell-O—CH₂—COO⁻Na⁺ (CMC)+H₂O mixture; and a solvent that is propylene glycol; where the molar concentration of the solute/solvent mixture is between approximately 0.55 mol/L and approximately 0.75 mol/L.

### CROSS-REFERENCE TO RELATED APPLICATIONS

The current application claims priority to U.S. Provisional Application No. 62/043,264, filed on Aug. 28, 2014, the disclosure of which is incorporated herein by reference.

### FIELD OF THE INVENTION

The present invention generally relates to thermal treatment packs.

### BACKGROUND

Thermal treatment packs are often used to treat muscle pain, joint pain, ligament pain, and/or body pain generally. For example, thermal treatment packs can be used to apply heat to reduce pain in joints and relax sore muscles, ligaments and tendons. The application of heat to the body for pain relief is often referred to as ‘heat therapy’. Thermal treatment packs can also be used to cool an afflicted body part so as to reduce blood flow to the body part, and correspondingly reduce pain and swelling. Cooling the body to reduce pain and swelling is often referred to as ‘cold therapy.’ In many instances, thermal treatment packs can be configured to apply either heat or cold to an anatomical region. For instance, many thermal treatment packs can either be heated (e.g. in a microwave) or cooled (e.g. in a freezer), and thereafter applied an afflicted anatomical region to provide either heat or cold therapy.

### SUMMARY OF THE INVENTION

Systems and methods in accordance with embodiments of the invention implement non-aggravating thermal treatment packs. In one embodiment, a non-aggravating thermal treatment pack includes: a vinyl-based encasing; and a gelatinous material disposed within the vinyl-based encasing, itself including: a solute that is a Cell-O—CH₂—COO⁻Na⁺ (CMC)+H₂O mixture; and a solvent that is propylene glycol; where the molar concentration of the solute/solvent mixture is between approximately 0.55 mol/L and approximately 0.75 mol/L.

In another embodiment, the molar concentration of the solute/solvent mixture is between approximately 0.60 mol/L and approximately 0.70 mol/L.

In yet another embodiment, the molar concentration of the solute/solvent mixture is approximately 0.65 mol/L.

In still another embodiment, the vinyl-based encasing conforms to a tubular eversion limitless geometry.

In still yet another embodiment, the vinyl-based encasing includes one of: polyvinyl chloride, polyvinyl fluoride, polyvinyl acetate, and mixtures thereof.

In a further embodiment, the vinyl based encasing is characterized by a thickness of between approximately 0.1 mm and approximately 0.6 mm.

In a yet further embodiment, the vinyl-based encasing is characterized by a Shore A durometer hardness value of between approximately 82 and approximately 88 shore A as measured in accordance with the ASTM D2240 standard.

In a still further embodiment, the vinyl-based encasing is characterized by a specific gravity of between approximately 1.09 and approximately 1.13.

In a still yet further embodiment, the vinyl-based encasing is characterized by a tensile strength of between approximately 280 Kg/cm² and approximately 320 Kg/cm².

In another embodiment, the vinyl-based encasing is characterized in that it is capable of withstanding elongation of approximately 500%.

In yet another embodiment, the vinyl-based encasing is characterized by a tear strength of between approximately 60 Kg/cm and approximately 100 Kg/cm.

In still another embodiment, the vinyl-based encasing conforms to a relatively more planar-shaped geometry.

In still yet another embodiment, a method of treating an anatomical region includes: preheating or precooling a non-aggravating thermal treatment pack that itself includes: a vinyl-based encasing; and a gelatinous material disposed within the vinyl-based encasing, itself including: a solute that is a Cell-O—CH₂—COO⁻Na⁺ (CMC)+H₂O mixture; and a solvent that is propylene glycol; where the molar concentration of the solute/solvent mixture is between approximately 0.55 mol/L and 0.75 mol/L; and

disposing the preheated or precooled non-aggravating thermal treatment pack proximate an anatomical region thereby providing it with hot therapy or cold therapy.

### BRIEF DESCRIPTION OF THE DRAWINGS

FIG. 1 illustrates a non-aggravating thermal treatment pack that is tubular and eversion-limitless in accordance with certain embodiments of the invention.

FIGS. 2A and 2B depict photographs of a non-aggravating thermal treatment that is tubular and eversion-limitless in accordance with an embodiment of the invention.

FIG. 3 illustrates a non-aggravating thermal treatment pack that includes fastening members in accordance with certain embodiments of the invention.

### DETAILED DESCRIPTION

Turning now to the drawings, systems and methods implementing non-aggravating thermal treatment packs are illustrated. While thermal treatment packs are known, conventional thermal treatment packs can cause frostbite, or ‘ice pack burn’, when used to cool an afflicted anatomical region. For example, in U.S. Pat. Nos. 7,060,086 and 7,621,944 to Wilson et al., tubular and eversion-limitless thermal treatment packs are described; however, the patents caution that the disclosed thermal treatment packs can cause frostbite, or ‘ice pack burn’. Accordingly, the patents disclose using a separate fabric (e.g. a sock, a sleeve, or an ACE bandage) to insulate the skin from the disclosed thermal treatment packs and to thereby deter the development of frostbite. The disclosures of U.S. Pat. Nos. 7,060,086 and 7,621,944 are hereby incorporated by reference in their entirety. In general, the cited patents allude to the discomfort that conventional thermal treatment packs can cause, and suggest incorporating a separate fabric to preempt this discomfort. However, as can be appreciated, it can be inconvenient to have to rely on an article separate from the thermal treatment pack when desiring cold therapy. Moreover, using an insulating fabric can lessen the beneficial effects of cold therapy provided by the thermal treatment pack as the fabric can absorb much of the cooling effects intended to have been provided to the user by the thermal treatment pack.

Accordingly, in many embodiments of the invention, non-aggravating thermal treatment packs are provided that can offer cold therapy with a reduced, if not eliminated, level of discomfort. For example, in many embodiments, thermal treatment packs are constructed from particular materials combinations that are unlikely to cause the development of condensation. In a number of embodiments, the thermal treatment packs further hinder the development of ‘ice pack burn’ or frostbite. For example, in a number of embodiments a thermal treatment pack includes a vinyl-based (e.g. PVC) encasing that contains a non-gaseous fluid. In numerous embodiments the non-gaseous fluid is gelatinous. In many embodiments, the gelatinous fluid includes a solute that is a Cell-O—CH₂—COO⁻Na⁺ (CMC)+H₂O mixture and an associated solvent that is propylene glycol. It has been discovered that this combination of constituent materials can surprisingly mitigate many of the uncomfortable experiences caused by conventional thermal treatment packs.

Notably, non-aggravating thermal treatment packs can adopt any geometry that can efficiently administer heat/cold therapy in accordance with many embodiments of the invention. For instance, in many embodiments, the thermal treatment packs adopt a tubular, eversion-limitless geometry. Non-aggravating thermal treatment packs are now discussed in greater detail below.

### Non-Aggravating Thermal Treatment Packs

In many embodiments, non-aggravating thermal treatment packs are provided. In numerous embodiments, the non-aggravating thermal treatment packs are characterized by a vinyl-based encasing that contains a non-gaseous fluid. The non-aggravating thermal pack can be heated (e.g. in a microwave) or cooled (e.g. in a freezer) as desired, and thereby be used to provide hot or cold therapy. In many embodiments, the non-gaseous fluid is gelatinous. In numerous embodiments, the gelatinous fluid includes a solute that is a Cell-O—CH2—COO⁻Na⁺ (CMC)+H₂O mixture and an associated solvent that is propylene glycol. Notably, non-aggravating thermal treatment packs can adopt any of a variety of shapes in accordance with many embodiments of the invention. For example, they can adopt a tubular, eversion-limitless shape, such as those disclosed in the above-cited patents. The tubular shape can be sized so as to accommodate a human appendage, e.g. a limb, or a finger. In this way, the tubular shape can be rolled along the appendage until it is sufficiently proximate the afflicted anatomical region so that it can thereby provide therapy. Note that rolling a tubular shape along an appendage can be a more comfortable way of disposing a thermal treatment pack proximate an afflicted anatomical region as compared to sliding the thermal treatment pack until it is proximate the anatomical region. For example, sliding a tubular shape along an appendage can cause frictional discomfort to the user. Accordingly, because frictional discomfort is not as much of a concern (if at all), tubular and eversion-limitless thermal treatment packs can be made to more tightly conform to the shape of a limb. In turn, this tight conformity with an appendage can allow the thermal treatment pack to more effectively provide localized hot/cold therapy. For example, a tightly conformed thermal treatment pack is more likely to stay affixed to the limb proximate the afflicted region, even where the user is moving the respective limb; no additional fastening members (e.g. a bandage or other wrap) may be needed.

Additionally, recall that a standard treatment protocol for healing a sprain is known by the acronym, ‘R.I.C.E.’: resting the sprain; icing the sprain (or otherwise cooling the sprain); compressing the sprain; and elevating the sprain. As can be inferred from the above, this protocol can be implemented using a tubular and eversion-limitless thermal treatment pack in accordance with embodiments of the invention. For example, a non-aggravating tubular and eversion-limitless thermal treatment pack in accordance with many embodiments can apply cold therapy under compression to a sprain. More specifically, as described above, a tubular and eversion-limitless thermal treatment pack can more tightly conform to the contours of a limb, and can thereby be made to apply cold therapy under compression.

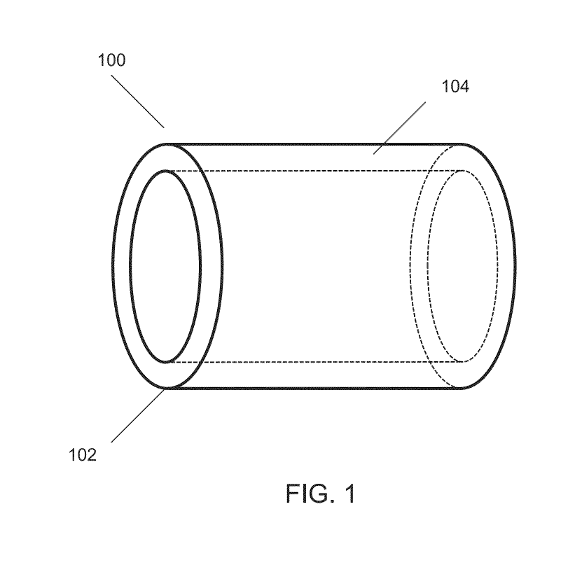

FIG. 1 depicts a non-aggravating thermal treatment pack that is tubular and eversion-limitless in accordance with an embodiment of the invention. In particular, a non-aggravating thermal treatment pack 100 is depicted that includes a vinyl-based encasing 102 that is tubular in shape. As alluded to previously, the tubular shape can be sized so as to accommodate a human appendage, e.g. a finger or a limb.

The vinyl-based encasing can be constructed from any suitable vinyl-based material in accordance with many embodiments of the invention. For example, in many embodiments, the vinyl-based encasing includes one of: polyvinyl chloride (PVC), polyvinyl fluoride (PVF), polyvinyl acetate (PVAc), and mixtures thereof. In many embodiments, the vinyl-based encasing is characterized by a Shore A durometer hardness value of between approximately 82 and approximately 88 shore A as measured in accordance with the ASTM D2240 standard. In numerous embodiments, the vinyl-based encasing is characterized by a specific gravity of between approximately 1.09 and approximately 1.13. In a number of embodiments, the vinyl-based encasing is characterized by a tensile strength of between approximately 280 Kg/cm² and approximately 320 Kg/cm² as measured in accordance with the ASTM D412 Standard. In several embodiments, the vinyl-based encasing is characterized by a tensile strength of approximately 300 Kg/cm² as measured in accordance with the ASTM D412 Standard. In many embodiments, the vinyl-based encasing can be elongated by approximately 500% as measured in accordance with the ASTM D412 standard. In several embodiments, the vinyl-based encasing is characterized by a tear strength of between approximately 60 Kg/cm and 100 kg/cm as measured in accordance with the ASTM D624 standard. In a number of embodiments, the vinyl-based encasing is characterized by a tear strength of approximately 80 Kg/cm. In a number of embodiments, the vinyl-based encasing has a thickness of between approximately 0.1 mm and approximately 0.6 mm. In many embodiments, the vinyl encasing is sufficiently pliable such that the tubular structure is eversion-limitless. While vinyl-based encasings having certain physical characteristics have been mentioned, it should be clear that any of a variety of vinyl-based encasings can be implemented in accordance with embodiments of the invention; the implemented vinyl-based encasings are not limited to those that conform to the above-mentioned properties.

Referring back to FIG. 1, it is illustrated that the vinyl-based encasing contains a non-gaseous fluid 104. The non-gaseous fluid can be any suitable non-gaseous fluid in accordance with many embodiments of the invention. In many embodiments, the non-gaseous fluid is gelatinous. In numerous embodiments, the gelatinous material is a combination of a solute that is a Cell-O—CH₂—COO⁻Na⁺ (CMC)+H₂O mixture and a solvent that is propylene glycol. In many instances the solute that is a Cell-O—CH₂—COO⁻Na⁺ (CMC)+H₂O mixture is itself a gel. Note that the ratio of the Cell-O—CH₂—COO⁻Na⁺ (CMC) to the H₂O can be any suitable ratio in accordance with embodiments of the invention. In general, Cell-O—CH₂—COO⁻Na⁺ (CMC) can be mixed with H₂O at a concentration of one of: 1% by weight; 2% by weight; 3% by weight; 4% by weight; 5% by weight; 6% by weight; 7% by weight; 8% by weight; 9% by weight; 10% by weight; 11% by weight; 12% by weight; 13% by weight; 14% by weight; 15% by weight; 16% by weight; 17% by weight; 18% by weight; 19% by weight; 20% by weight; 21% by weight; 22% by weight; 23% by weight; 24% by weight; 25% by weight; 26% by weight; 27% by weight; 28% by weight; 29% by weight; 30% by weight; 31% by weight; 32% by weight; 33% by weight; 34% by weight; 35% by weight; 36% by weight; 37% by weight; 38% by weight; 39% by weight; 40% by weight; 41% by weight; 42% by weight; 43% by weight; 44% by weight; 45% by weight; 46% by weight; 47% by weight; 48% by weight; 49% by weight; 50% by weight; 51% by weight; 52% by weight; 53% by weight; 54% by weight; 55% by weight; 56% by weight; 57% by weight; 58% by weight; 59% by weight; 60% by weight; 61% by weight; 62% by weight; 63% by weight; 64% by weight; 65% by weight; 66% by weight; 67% by weight; 68% by weight; 69% by weight; 70% by weight; 71% by weight; 72% by weight; 73% by weight; 74% by weight; 75% by weight; 76% by weight; 77% by weight; 78% by weight; 79% by weight; 80% by weight; 81% by weight; 82% by weight; 83% by weight; 84% by weight; 85% by weight; 86% by weight; 87% by weight; 88% by weight; 89% by weight; 90% by weight; 91% by weight; 92% by weight; 93% by weight; 94% by weight; 95% by weight; 96% by weight; 97% by weight; 98% by weight; and 99% by weight. In general, any of a variety of suitable mixture concentrations can be implemented.

Additionally, while Cell-O—CH₂—COO⁻Na⁺ (CMC) is referenced, in many instances, other water-soluble salts of carboxymethyl cellulose are implemented. For instance, in some embodiments, the solute is one of a potassium carboxymethyl cellulose—H₂O mixture and an ammonium carboxymethyl cellulose—H₂O mixture. Any suitable water soluble salt of carboxymethyl cellulose can be implemented in accordance with certain embodiments of the invention.

In several instances the molar concentration of the solute/solvent mixture is between approximately 0.55 mol/L and approximately 0.75 mol/L. In a number of embodiments, the molar concentration of the solute/solvent mixture is between approximately 0.60 mol/L and approximately 0.70 mol/L. In many embodiments, the molar concentration of the solute/solvent mixture is approximately 0.65 mol/L. The combination of a vinyl-based encasing as described above in conjunction with this gelatinous material—and in particular, the Cell-O—CH2—COO⁻Na⁺ (CMC)-based gelatinous material—has been surprisingly demonstrated to be particularly effective at reducing occurrences of discomfort during cold therapy. It is believed that this combination is effective for inhibiting condensation/dripping and is thereby effective at inhibiting potential skin burn. Although, it should be clear that embodiments of the invention are not limited by this proposed theory that the discussed combinations tend to inhibit condensation/dripping and thereby inhibit potential skin burn. Rather, this theory is being offered gratuitously as a possible explanation for the observed phenomena that discomfort is reduced with the disclosed material combinations. As can be appreciated, in many instances the non-gaseous fluid can be capable of providing heat therapy or cold therapy. For example, the non-gaseous fluid can be heated (e.g. via microwave) so that it can provide heat therapy or can be cooled (e.g. via a freezer) so that it can provide cold therapy.

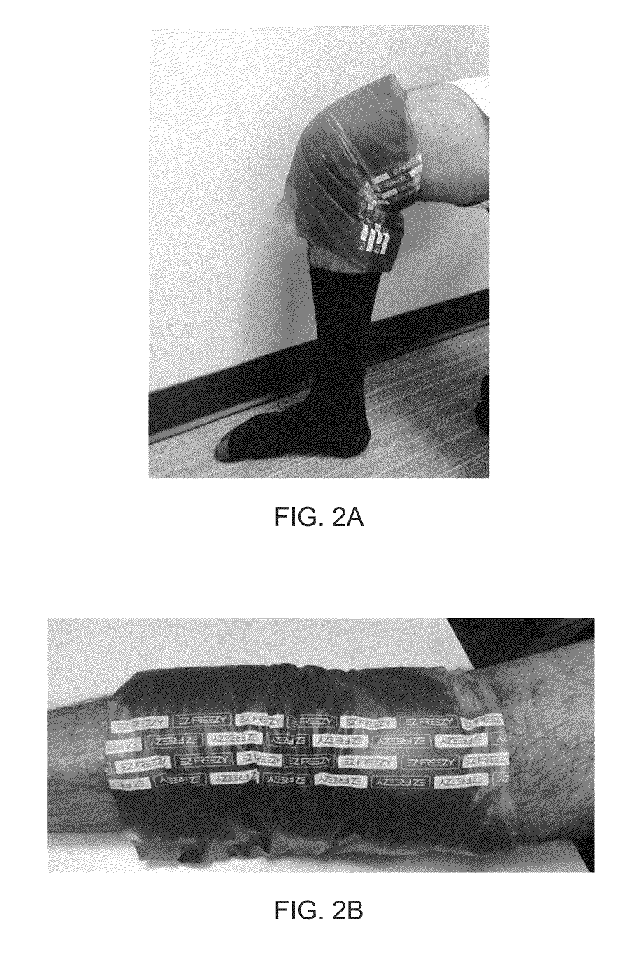

FIGS. 2A and 2B are photographs of a non-aggravating thermal treatment pack that is tubular and eversion limitless in accordance with an embodiment of the invention. In particular, the depicted non-aggravating thermal treatment pack is sized to accommodate a leg. Of course, it should be clear that non-aggravating thermal treatment packs can be sized to accommodate any limb or appendage in accordance with embodiments of the invention.

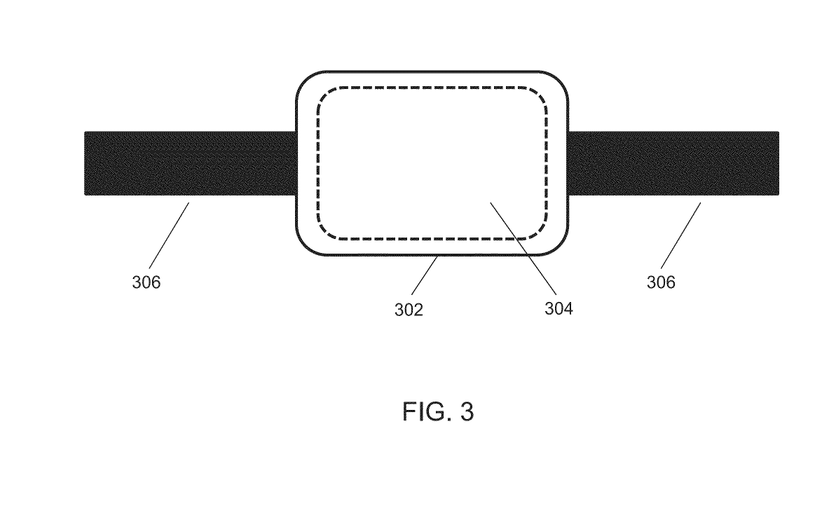

Although a tubular eversion-limitless has been described above and illustrated in FIGS. 1 and 2A-2B, it should be clear that non-aggravating thermal treatment packs can be implemented via any suitable shape in accordance with many embodiments of the invention. For instance, in some embodiments, the vinyl-based encasing is relatively more planar, and can be coupled to fastening members that can allow the vinyl-based encasing to be temporarily affixed to an anatomical region. For example, FIG. 3 depicts a thermal treatment pack that is relatively more planar and includes straps for fastening the thermal treatment pack to an in accordance with certain embodiments of the invention. In particular, the thermal treatment pack 300 includes a vinyl-based encasing that is relatively more planar in shape 302 and contains a non-gaseous fluid 304, e.g. a gelatinous material. The thermal treatment pack 300 also includes straps 306 for fastening the vinyl-based encasing to an anatomical region, e.g. an appendage or some portion of the torso, so that it can administer thermal treatment. Non-aggravating thermal treatment packs that include distinct fastening members may be particularly well-suited to provide hot/cold therapy to regions of the torso—whereas tubular and eversion-limitless geometries may not be able to easily access the torso, a non-aggravating thermal treatment pack may be able to be more easily affixed to regions of the torso.

Of course, while thermal treatment packs have been described for treating certain anatomical regions, it should of course be understood that thermal treatment packs can be adapted to treat any of a variety of parts of the human body in accordance with many embodiments of the invention. For example, in many embodiments, thermal treatment packs are adapted so that they can provide hot/cold therapy to the neck area.

Additionally, as can be appreciated, many embodiments of the invention are directed towards methods for treating an anatomical region using any of the above-described non-aggravating thermal treatment packs. In particular, many embodiments involve preheating or precooling a non-aggravating thermal treatment pack according to any of the configurations disclosed above, and disposing the preheated or precooled non-aggravating thermal treatment pack proximate an anatomical region for which hot or cold therapy is desired. As can be appreciated, any of the above-disclosed non-aggravating thermal treatment packs can be implemented. For example, in many embodiments, non-aggravating thermal treatment packs conforming to a tubular eversion limitless geometry as described above are implemented. In a number of embodiments, non-aggravating thermal treatment packs that conform to more of a planar geometry are implemented. In many embodiments, the implemented non-aggravating thermal treatment pack includes a vinyl-based encasing, and gelatinous fluid that includes a solute that is a Cell-O—CH₂—COO⁻Na⁺ (CMC)+H₂O mixture and an associated solvent that is propylene glycol, where the molar concentration of the solute/solvent mixture is between approximately 0.55 mol/L and approximately 0.75 mol/L. Of course, it should be clear that any of the above-described non-aggravating thermal treatment packs can be implemented in accordance with embodiments of the invention.

While certain geometries have been discussed, non-aggravating thermal treatment packs can be implemented in any suitable shape that can allow for efficient hot/cold therapy in accordance with many embodiments of the invention. More generally, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. A non-aggravating thermal treatment pack comprising:
a vinyl-based encasing; and
a gelatinous material disposed within the vinyl-based encasing comprising:
a solute that is a Cell-O—CH₂—COO⁻Na⁺ (CMC)+H₂O mixture; and
a solvent that is propylene glycol;
wherein the molar concentration of the solute/solvent mixture is between approximately 0.55 mol/L and approximately 0.75 mol/L.

2. The non-aggravating thermal treatment pack of claim 1, wherein the molar concentration of the solute/solvent mixture is between approximately 0.60 mol/L and approximately 0.70 mol/L.

3. The non-aggravating thermal treatment pack of claim 2, wherein the molar concentration of the solute/solvent mixture is approximately 0.65 mol/L.

4. The non-aggravating thermal treatment pack of claim 1, wherein the vinyl-based encasing conforms to a tubular eversion limitless geometry.

5. The non-aggravating thermal treatment pack of claim 1, wherein the vinyl-based encasing comprises one of: polyvinyl chloride, polyvinyl fluoride, polyvinyl acetate, and mixtures thereof.

6. The non-aggravating thermal treatment pack of claim 5, wherein the vinyl based encasing is characterized by a thickness of between approximately 0.1 mm and approximately 0.6 mm.

7. The non-aggravating thermal treatment pack of claim 6, wherein the vinyl-based encasing is characterized by a Shore A durometer hardness value of between approximately 82 and approximately 88 shore A as measured in accordance with the ASTM D2240 standard.

8. The non-aggravating thermal treatment pack of claim 6, wherein the vinyl-based encasing is characterized by a specific gravity of between approximately 1.09 and approximately 1.13.

9. The non-aggravating thermal treatment pack of claim 6, wherein the vinyl-based encasing is characterized by a tensile strength of between approximately 280 Kg/cm² and approximately 320 Kg/cm².

10. The non-aggravating thermal treatment pack of claim 6, wherein the vinyl-based encasing is characterized in that it is capable of withstanding elongation of approximately 500%.

11. The non-aggravating thermal treatment pack of claim 6, wherein the vinyl-based encasing is characterized by a tear strength of between approximately 60 Kg/cm and approximately 100 Kg/cm.

12. The non-aggravating thermal treatment pack of claim 1, wherein the vinyl-based encasing conforms to a relatively more planar-shaped geometry.

13. A method of treating an anatomical region comprising:
preheating or precooling a non-aggravating thermal treatment pack that comprises:
a vinyl-based encasing; and
a gelatinous material disposed within the vinyl-based encasing, itself comprising:
a solute that is a Cell-O—CH₂—COO⁻Na⁺ (CMC)+H₂O mixture; and
a solvent that is propylene glycol;
wherein the molar concentration of the solute/solvent mixture is between approximately 0.55 mol/L and 0.75 mol/L; and
disposing the preheated or precooled non-aggravating thermal treatment pack proximate an anatomical region thereby providing it with hot therapy or cold therapy.
